(19) United States
(12) Patent Application Publication
(10) Pub. No.: US 2025/0082261 A1
(43) Pub. Date: Mar. 13, 2025

(54) DEVICES, SYSTEMS, AND METHODS FOR TRACKING AND ASSESSING A WOUND

(71) Applicant: Hill-Rom Services, Inc., Batesville, IN (US)

(72) Inventors: Angela J. Williams, Batesville, IN (US); Christopher Nelson, Batesville, IN (US); Harsh Dweep, Batesville, IN (US)

(73) Assignee: Hill-Rom Services, Inc., Batesville, IN (US)

(21) Appl. No.: 18/882,132

(22) Filed: Sep. 11, 2024

Related U.S. Application Data
(60) Provisional application No. 63/582,027, filed on Sep. 12, 2023.

(51) Int. Cl. A61B 5/00; A61B 5/01

(52) U.S. Cl. CPC A61B 5/445; A61B 5/0077; A61B 5/01; A61B 5/742

(57) ABSTRACT

A method for tracking and assessing a wound includes identifying a wound from an image output by a camera, identifying a set of wound features based on the image, detecting a temperature of the wound from at least one heat sensor, triggering at least one response if the wound temperature is outside of a therapeutic temperature zone, processing the set of wound features and wound temperature using machine learning, determining an optimal wound healing trajectory based on the set of wound features and the wound temperature using machine learning, predicting at least one future wound condition based on the optimal wound healing trajectory using machine learning, and displaying the optimal wound healing trajectory and the at least one future wound condition on a display.

### CROSS-REFERENCE TO RELATED APPLICATIONS

This application claims priority to U.S. Provisional Patent Application Ser. No. 63/582,027, filed Sep. 12, 2023, entitled, “DEVICES, SYSTEMS, AND METHODS FOR TRACKING AND ASSESSING A WOUND,” the entirety of which is incorporated by reference herein.

### FIELD

The present disclosure generally relates to wound monitoring, and more particularly, to devices, systems, and methods to track and assess a wound of a subject objectively.

### BACKGROUND

Subjects can suffer from various types of wounds that may or may not require hospital admission. Changes in wound characteristics is a laborious task which many clinicians may struggle to find time to adequately document. Various solutions have been presented to automate the task of documenting changes in wound characteristics. However, these solutions may not adequately consider wound temperature and its effect on optimal wound healing trajectories. In addition, these solutions fail to provide a mechanism for shortening the optimal wound healing trajectory. Accordingly, it may be desirable to consider wound temperature and mechanisms for shortening optimal wound healing trajectories in automating the task of documenting changes in wound characteristics.

### SUMMARY

In one aspect, a method for tracking and assessing a wound may include identifying a wound from an image output by a camera, identifying a set of wound features based on the image, detecting a temperature of the wound from at least one heat sensor, triggering at least one response if the wound temperature is outside of a therapeutic temperature zone, and processing the set of wound features and wound temperature using machine learning. The method may further include determining an optimal wound healing trajectory based on the set of wound features and the wound temperature using machine learning, predicting at least one future wound condition based on the optimal wound healing trajectory using machine learning, and displaying the optimal wound healing trajectory and the at least one future wound condition on a display.

In another aspect, a system for tracking and assessing a wound may include a display, a camera configured to output image data, at least one heat sensor configured to output temperature data, a memory containing a machine readable medium including machine executable code having instructions stored thereon, and a controller communicatively coupled to the display, camera, at least one heat sensor, and memory. The controller includes one or more processors and is configured to execute the machine executable code to cause the controller to: identify a wound and a set of wound features from the image data, detect a temperature of the wound from the at least one heat sensor, trigger at least one response if the wound temperature is outside of a therapeutic temperature zone, process the set of wound features and wound temperature using machine learning, determine an optimal wound healing trajectory based on the set of wound features and the wound temperature using machine learning, predict at least one future wound condition based on the optimal wound healing trajectory using machine learning, and display the optimal wound healing trajectory and the at least one future wound condition on the display.

In another aspect, a method for tracking and assessing a burn wound may include identifying a burn wound from an image output by a camera, capturing a set of burn wound features based on the image, triaging the burn wound based on sensor data from one or more sensors, processing the burn wound features and sensor data with a machine learning algorithm to determine an optimal burn wound healing trajectory, wherein the machine learning algorithm is trained with a library of burn wound statistics, predicting at least one future burn wound condition based on the optimal wound healing trajectory with the machine learning algorithm, and displaying the optimal burn wound healing trajectory and the at least one future burn wound condition on a display.

In yet another aspect, a non-transitory, processor-readable storage medium, may include one or more programming instructions stored thereon, the one or more programming instructions, when executed, causing a processing device to carry out a method for tracking and assessing a wound and/or a burn wound.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRA WINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

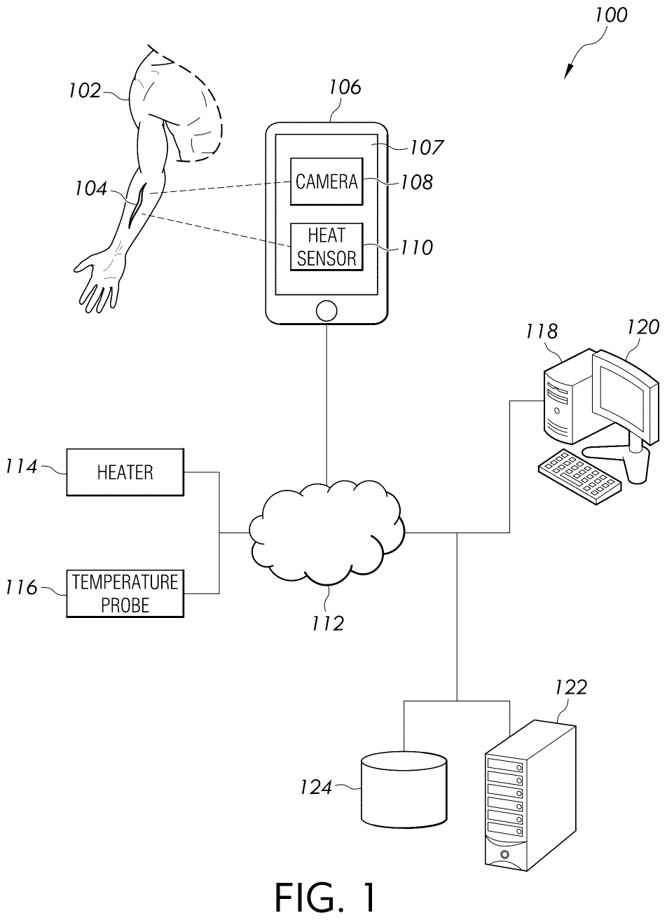

FIG. 1 schematically depicts an example system for tracking and assessing a wound, according to one or more embodiments shown and described herein;

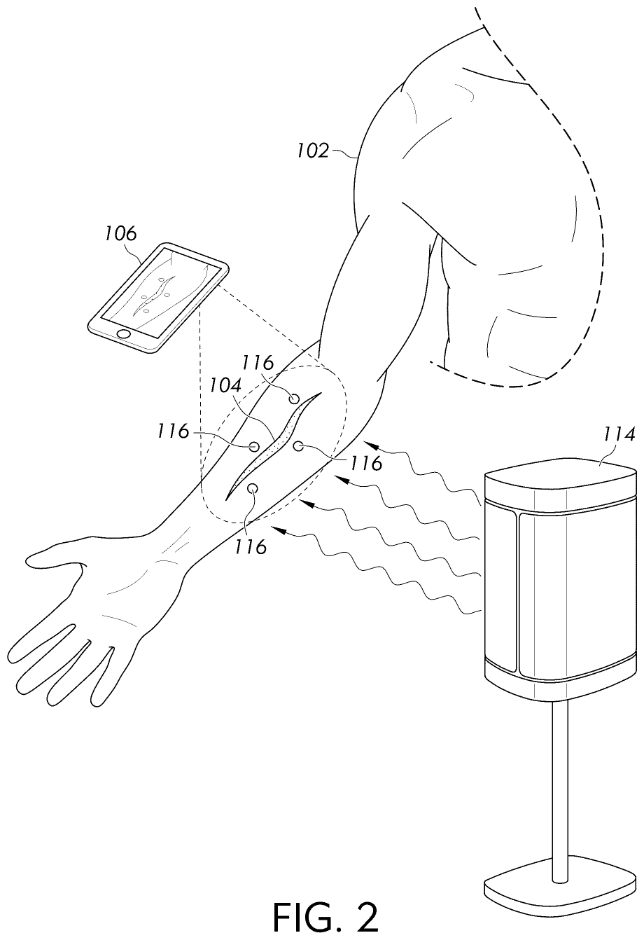

FIG. 2 schematically depicts an example smart device and heater used in the implementation of a method for tracking and assessing a wound, according to one or more embodiments shown and described herein;

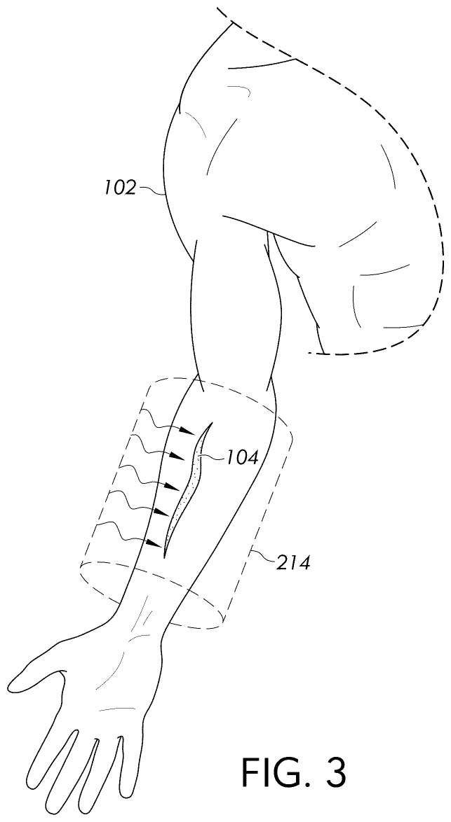

FIG. 3 schematically depicts an example heater used in the implementation of a method for tracking and assessing a wound, according to one or more embodiments shown and described herein;

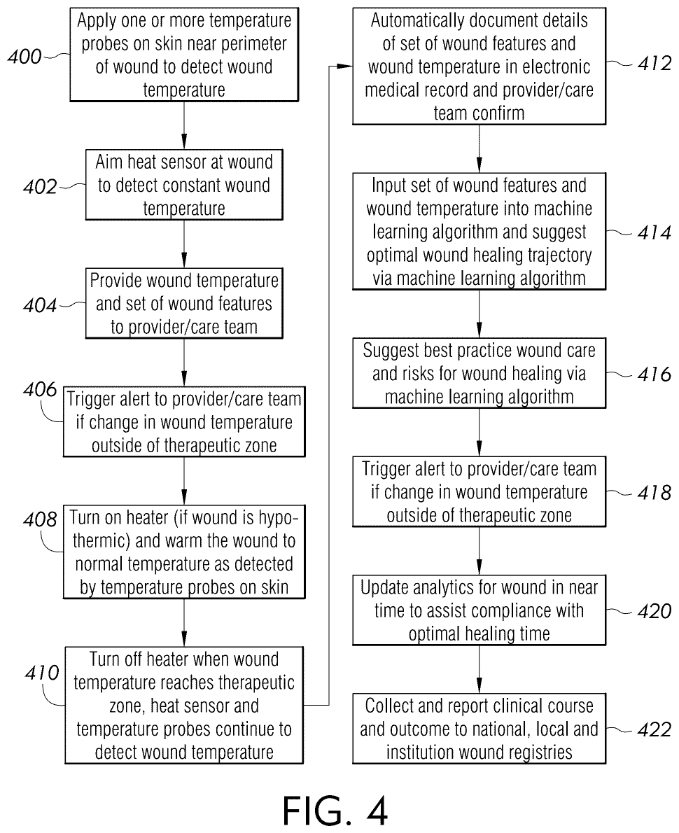

FIG. 4 depicts a flow diagram of an example method to track and assess a wound, according to one or more embodiments shown and described herein; and

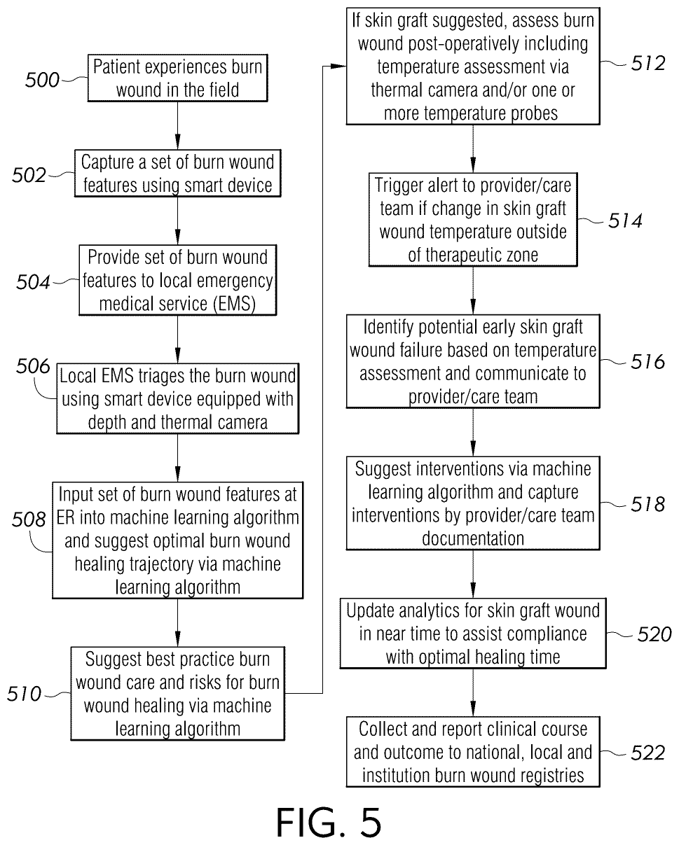

FIG. 5 depicts a flow diagram of an example method to track and assess a burn wound, according to one or more embodiments shown and described herein.

### DETAILED DESCRIPTION

The present disclosure relates to devices, systems, and methods to objectively track and assess a wound. In particular, a wound is identified from an image and a set of wound features is identified based on the image. A temperature of the wound is detected in real time and a response is triggered if the wound temperature is outside of a therapeutic temperature zone. In particular, the response may include activating a heater to warm the wound. The set of wound features and wound temperature are input into a machine learning algorithm for processing using machine learning, and an optimal wound healing trajectory is determined. Real time temperature detection improves existing wound monitoring systems because decisions affecting wound healing can be made more quickly. The ability to warm the wound using heater improves existing wound monitoring systems by providing a mechanism to shorten an optimal wound healing trajectory.

As discussed above, a wound is identified from an image and a set of wound features is identified based on the image. A temperature of the wound is detected in real time and a response is triggered if the wound temperature is outside of a therapeutic temperature zone. The response may include activating a heater to warm the wound. The ability to warm the wound using a heater improves existing wound monitoring systems by providing a mechanism to shorten an optimal wound healing trajectory. The set of wound features and wound temperature are input into a machine learning algorithm for processing using machine learning, and the optimal wound healing trajectory is determined. The machine learning algorithm is trained on a library of wound statistics. Use of the library of wound statistics improves upon existing wound monitoring systems because the accuracy of determining the optimal wound healing trajectory improves over time. Moreover, the devices, systems, and methods disclosed herein objectively track and assess a wound because the tracking and assessing is based on objective factors. For example, the tracking and assessing described herein may be based on wound location, size, shape, color, depth, temperature, humidity, etc.

Turning now to the figures, FIG. 1 illustrates a wound tracking and assessing system 100, according to one or more embodiments of the present disclosure. The system 100 includes a smart device 106 with a camera 108 and a heat sensor 110 that is usable (e.g., arranged, positioned, or the like) to image a wound 104 on a subject 102. The smart device 106 may be communicatively coupled to a wired or wireless network 112 and may transmit image and/or temperature data from the camera 108 and heat sensor 110 over the network 112. As used herein, the term “temperature data” may further include humidity data. The network 112 may be communicatively coupled to other smart devices such as, for example, a heater 114 and/or one or more temperature probes 116. The network 112 may further be communicatively coupled to other computing devices such as, for example, a computer 118 and/or one or more displays 120. The network 112 may also be communicatively coupled to a remote server 122 and a database 124. Accordingly, the smart device 106, including camera 108 and heat sensor 110, the heater 114, the one or more temperature probes 116, the computer 118 and one or more displays 120, and the server 122 and database 124 may each be communicatively coupled to one another via network 112.

The wound 104 on the subject 102 may be any kind of wound, including a wound to the skin or a wound that may be visible with the camera 108 or heat sensor 110. For example, the wound 104 may be a penetrating wound such as a puncture wound, surgical wound/incision, thermal, chemical, or electrical burns, bites and stings, and/or a gunshot wound. The wound 104 may also be from blunt force trauma, such as an abrasion, laceration, or skin tear. The wound 104 may further be a closed wound, such as a contusion, blister, seroma, hematoma, or crush injury. The wound 104 may also additionally be a skin ulcer. The wound 104 may be of different sizes or colors and may be at any stage of the healing process. In some instances, the wound 104 may not be visible to the naked eye (e.g., topical), but may be capable of being imaged using the devices described herein (e.g., a subdermal wound that can be captured/detected via infrared imaging or the like). Moreover, while the wound 104 is illustrated in FIGS. 1-3 as being located on an arm of the subject 102, it should be understood that the wound 104 may be located on any part of the subject's body without departing from the scope of the present disclosure.

The smart device 106 may be a cell phone, tablet, or other suitable computing device. In some embodiments, the smart device 106 is a controller that controls the operation of the system 100. In this regard, the smart device 106 includes a processor and a non-transitory memory module that are communicatively coupled to one another, wherein the non-transitory memory module of the smart device 106 includes computer readable and executable instructions that may be executed by the processor of the smart device 106. Also, the smart device 106 may include a touch screen display 107 for outputting data for viewing by the subject 102 and/or care team. The smart device 106 may transmit image data from camera 108, temperature and/or humidity data from the heat sensor 110, and other data related to the wound over the network 112 (e.g., via communications hardware (not depicted)). The smart device 106 may further include a graphical user interface (GUI) for inputting data by the subject 102 and/or care team and for controlling the operation of the various components of the system 100. That is, the initialization of system 100 may be achieved via the smart device 106.

The camera 108 is configured to capture images and/or video of the wound 104 and may include a plurality of cameras that work together or independently to capture images from various points of view. The heat sensor 110 may be included as part of camera 108 or separate and external from the camera 108. Thus, the camera 108 and/or heat sensor 110 are configured to capture images in any electromagnetic frequency range, including the visible spectrum and the infrared (IR) spectrum. The camera 108 may include various types of cameras and/or optical sensors. For example, the camera 108 may include various types of optical sensors, including RGB, LIDAR optical sensors, or a combination thereof. The heat sensor 110 may include a thermal camera or IR sensor, for example (e.g., a camera module provided by Teledyne FLIR (Wilsonville, OR)). Furthermore, the heat sensor 110 may be configured to determine surface humidity. In embodiments, the camera 108 may be configured to capture still images and/or video. The camera 108 may capture still images, a plurality of images over a predetermined time period, and/or video. The heat sensor 110 is configured to detect, in real time, a temperature and/or surface humidity of the wound. As used herein, “real time” refers to the capability of the heat sensor 110 (or other device) to provide instantaneous and continuous updates of the temperature and/or surface humidity as it changes. As used herein, “real time” can include responses within a given latency, which can vary from sub-second to seconds.

The network 112 may be configured as any wired and/or wireless network such as, for example, wide area networks, the internet, an intranet, or the like. The network may further be configured with hardware, including any device capable of transmitting and/or receiving data via wired or wireless network 112. For example, the hardware of network 112 can include a chipset (e.g., antenna, processors, machine readable instructions, etc.) to communicate over wired and/or wireless computer networks such as, for example, wireless fidelity (Wi-Fi), WiMax, Bluetooth, or the like.

The heater 114 is configured to supply at least one type of heat to the wound 104. For example, the heater 114 may supply radiant heat and/or convection heat to the wound 104. Devices that supply radiant heat, such as heater 114 in some embodiments, are generally configured to emit heat in the form of electromagnetic waves, which travel directly and heat up objects and surfaces without needing a medium like air to carry the heat. Devices that supply convection heat, such as heater 114 in some embodiments, are generally configured to transfer heat through the movement of fluids caused by the differences in density and temperature within the fluid. For example, heater 114 may include a heating element and a fan to quickly distribute warm air. In some embodiments, such as the embodiment illustrated in FIG. 2, the heater 114 may be mounted to a stand that is capable of being placed close to the wound 104. However, the heater 114 described herein is not limited to being mounted to a stand. That is, in other embodiments, the heater 114 may be mounted to another component or may be handheld such that the heater 114 is permitted to be positioned close to the wound 104. For example, the heater 114 may be mounted to a bed or chair on which the subject 102 can lay or be seated. The heater 114 may also be attached to an articulating arm or boom mounted to the ceiling or wall which permits positioning of the heater 114 in different locations and orientations relative to the wound 104 of subject 102. Alternatively, the heater 114 could also be mounted directly to the ceiling or wall.

With reference to FIG. 3, in some embodiments, the heater 214 may be mounted to the body of subject 102. That is, the heater 214 may be configured as an electrically powered heating sleeve, wrap, blanket, sheath, or the like which can be fitted to the body of subject 102 at a location where wound 104 is located. Similar to heater 114, the body-mounted heater 214 may be configured to supply at least one of radiant and/or convection heat to the wound 104. In some embodiments, the body-mounted heater 214 may further be configured with a moisturizing device (not shown) which can be activated to supply moisture to the wound 104 through the application of water or a sterile/aseptic liquid suitable for application to wound 104, for example. Heaters 114, 214 may be powered by alternating current (AC) or direct current (DC).

In some embodiments, the heaters 114, 214 may be provided with a chipset (e.g., antenna, processors, machine readable instructions, etc.) to communicate over network 112, thereby permitting remote control of the heaters 114, 214. For example, the smart device 106 and/or computer 118 may be used to control the heaters 114, 214.

The one or more temperature probes 116 may be used in conjunction with the heat sensor 110 to obtain accurate temperature measurements of the wound 104. Furthermore, the one or more temperature probes 116 may be configured to detect surface humidity of the wound 104. In this regard, as shown in FIG. 2, the one or more temperature probes 116 are generally placed around a perimeter of the wound 104, as close as possible to the wound bed without being placed directly in the wound bed, to monitor and measure the temperature and/or humidity of the wound bed in real time. The perimeter of the wound 104 is generally defined as a path that encompasses, surrounds, or outlines the two-dimensional shape of the wound on the subject's 102 skin. Determination of the wound perimeter may be made based on a visual inspection of the color change between typical wound colors, hues, or textures and the native hue of the subject's 102 skin. Determination of the wound perimeter may also be made based on a visual inspection of the height change between the wound 104 and the subject's 102 surrounding skin, where the wound 104 may be raised or recessed with respect to the normal plane along which the subject's 102 skin extends. As discussed in further detail below, the system 100 itself may be configured to determine the perimeter of the wound 104 and inform placement of the one or more temperature probes 116 at the perimeter of the wound 104. Moreover, the one or more temperature probes 116 may also be configured with a chipset (e.g., antenna, processors, machine readable instructions, etc.) to communicate temperature and/or humidity readings over network 112.

The computer 118 may be any suitable computing device that generally includes a processor and a non-transitory memory module that are communicatively coupled to one another, wherein the non-transitory memory module of the computer 118 includes computer readable and executable instructions that may be executed by the processor of the computer 118. As such, in some embodiments, the computer 118 is a controller that controls the operation of the system 100. In some embodiments, the computer 118 may control the system 100 in addition to or alternatively from the smart device 106. The computer 118 also includes one or more displays 120 for outputting data for viewing by the subject and/or care team. The computer 118 may further include a GUI for inputting data by the subject 102 and/or care team and for controlling the operation of the various components of the system 100. That is, the initialization of system 100 may also be achieved via the computer 118.

In embodiments, the system 100 may be configured to establish a network connection so as to communicate with one or more remote servers 122. For example, the smart device 106 or computer 118 may communicate wireless or via a wired connection with one or more remote servers 122. In this regard, data from the camera 108 and/or heat sensor 110 may be remotely streamed on a device/display associated with the remote server 122.

In embodiments, the system 100 may be configured to establish a network connection so as to communicate with one or more databases 124. For example, the smart device 106 or computer 118 may communicate wireless or via a wired connection with the database 124. In this regard, data from the camera 108 and/or heat sensor 110 may be remotely stored on the database 124. Also, depending on the nature of the algorithms stored in the memory of the smart device 106 and/or computer 118, the algorithms may generate output and such output may be remotely stored on the database 124.

A method for tracking and assessing a wound 104 using system 100 discussed above will now be described with reference to FIG. 4. In some embodiments, various steps of the method may be performed by smart device 106, computer 118, and/or remote server 122. The various steps may be performed on separate computing devices and control systems or the steps may all be performed by a single processor. For example, the processing of image and temperature data may take place on the smart device 106, the computer 118, or on the remote server 122. As another example, certain portions of the processing that require heavier CPU load may take place on the remote server 122. As an additional example, scaled down versions of machine learning algorithms may run locally on the smart device 106 or computer 118.

At block 400, the one or more temperature probes 116 are applied on the skin of subject 102 near the perimeter of the wound 104 to aid in detecting the temperature and/or humidity of the wound 104. The one or more temperature probes 116 transmit temperature data in real time over the network 112 and the temperature and/or humidity is received by the smart device 106, computer 118, and/or server 122 of system 100. In addition, the temperature data from the one or more temperature probes 116 may be stored in the memory of the smart device 106 and/or the memory of the computer 118. Use of the one or more temperature probes 116 advantageously provides temperature and/or humidity monitoring that is more accurate compared to use of a heat sensor (e.g., heat sensor 110) alone. Moreover, real time temperature and/or humidity detection by the one or more temperature probes 116 (or by the heat sensor 110) described herein improves existing wound monitoring systems because decisions affecting wound healing can be made more quickly. For example, the decision to activate the heater 114, 214, as described in further detail below, can be made more quickly to ensure that the wound 104 maintains a temperature within the therapeutic temperature zone.

The method of FIG. 4 continues at block 402, where the heat sensor 110 is aimed at the wound 104 to detect a constant wound temperature and/or humidity. The heat sensor 110 transmits temperature data, including humidity data, in real time over the network 112 and the temperature data may be received by the smart device 106, computer 118, and/or server 122 of system 100. In addition, the temperature data from the heat sensor 110 may be stored in the memory of the smart device 106 and/or the memory of the computer 118.

At block 404, the wound temperature data from the one or more temperature probes 116 and the heat sensor 110 are provided to the provider/care team of subject 102. For example, the wound temperature data may be transmitted to the smart device 106 or computer 118 such that the care team of subject 102 can view the wound temperature data on the display 107 of the smart device 106 or the display 120 of the computer 118. The wound temperature data may also be stored in the memory of the smart device 106 and/or the memory of the computer 118 for subsequent retrieval, manipulation, and/or management.

Block 404 also includes providing a set of wound features to the provider/care team of subject 102. To obtain the set of wound features, the system 100 identifies the wound 104 from an image output by the camera 108. Thus, the subject 102 or member of the subject's care team may capture an image of the wound 104 using camera 108 of smart device 106 and the image may be received by the processor of the smart device 106. The image may additionally or alternatively be transmitted over network 112 and received by the processor of the computer 118. This image may include a color image and/or may include temperature data output by the heat sensor 110 and/or one or more temperature probes 116. The system 100 may process the image to identify wound boundaries. That is, the processor of smart device 106 or computer 118 may process the image to identify the wound boundaries. This may include determining the locations of pixels that represent a perimeter of the wound 104. In some embodiments, a color change threshold from typical wound colors, hues or textures, to a native hue of a subject's 102 skin may indicate the boundaries of wound 104. In other embodiments, changes in height or depth detected by LIDAR or similar sensors may be utilized to detect the boundaries of the wound 104. In additional embodiments, various computer vision processing techniques are available to identify the wound 104 and its boundaries as known to those of skill in the art. Additionally, temperature and humidity measurements from the heat sensor 110 and/or one or more temperature probes 116 may be mapped to the wound boundaries to determine which measurements output from the heat sensor 110 and/or one or more temperature probes 116 are within the wound boundaries.

Once the wound 104 is identified by the image output by camera 108, the set of wound features within the wound boundaries are obtained by the system 100 based on the image. The set of wound features may include features such as temperature and/or humidity (as output by the heat sensor 110 and/or one or more temperature probes 116), size, shape, color, and/or depth. The system 100 may determine the temperature and/or humidity features based on the output of the heat sensor 110 and/or one or more temperature probes 116 and may be determined in combination with the wound boundary information from the color images. The system 100 may also determine the size and shape (e.g., round, oval, irregular, or linear) of the wound 104 based on the image data using one or more algorithms known to those of skill in the art. The system 100 may also determine the color of the wound 104, including various other optical features of the images of the wound 104 output by camera 108. Various colors may indicate or relate to various stages of healing of the wound 104. Additionally, various colors may indicate inflammation, or infection of the wound 104. For example, red may indicate granulation tissue, yellow may indicate slough, and black may indicate eschar or necrotic tissue. The system 100 may further determine the depth of the wound 104, including superficial, partial-thickness, or full-thickness involvement based on the image data output by the camera 108, which may include a LIDAR sensor or other suitable sensor. The system 100 may further determine location of the wound 104 on the subject's 102 body. For example, the location may be determined as an area of bony prominence or high friction. The system 100 may further determine edge characteristics of the wound 104 such as maceration, undermining, or rolled edges. The system may further determine the presence, type, and amount of wound drainage or exudate, which can be can be serous (clear), sanguineous (bloody), serosanguineous (clear and bloody), purulent (yellow, green, or brown), or mixed.

The set of wound features thus being obtained as described above are provided to the provider/care team at block 404. For example, the set of wound features are transmitted to the smart device 106 or computer 118 such that the care team of subject 102 can view the set of wound features on the display 107 of the smart device 106 or the display 120 of the computer 118. The set of wound features may also be stored in the memory of the smart device 106 and/or the memory of the computer 118 for subsequent retrieval, manipulation, and/or management. For example, the set of wound features may be used in a subsequent step to determine an optimal wound healing trajectory, as discussed in further detail below.

As mentioned above, the system 100 itself may be configured to determine the perimeter or boundaries of the wound 104 and inform placement of the one or more temperature probes 116 at the perimeter/boundaries of the wound 104. In this regard, the system 100 identifies the perimeter/boundaries of the wound 104 and the set of wound features from an image output by the camera 108 as described above. Having obtained the perimeter/boundaries and the set of features of the wound 104, the system 100 may label the image with suggested locations for placement of the one or more temperature probes 116, taking into account the perimeter/boundaries and the set of wound features, and display the labeled image on the smart device 106 or computer 118. For example, a perimeter of wound 104 may be identified and a placement location for the one or more temperature probes 116 on the perimeter may be selected based on the proximity of the placement location to an identified feature of the wound. For example, a placement location may be selected on the wound perimeter based on an identified maximum diameter, width, or depth of the wound so long as the placement location does not coincide with an identified area of infection to prevent further disturbance/infection at that area. As another example, the system 100 may also suggest a specific number of temperature probes 116 to place on the wound perimeter based on the identified size of the wound and the set of wound features. In this regard, the system 100 is configured to strategically suggest placement locations of the one or more temperature probes 116 in a manner that does not hinder or is unlikely to hinder the healing of the wound 104.

At block 406, the system 100 triggers at least one response if the wound temperature is outside of a therapeutic temperature zone. In some embodiments, the at least one response includes sending an alert if the wound temperature is outside of the therapeutic temperature zone. The alert may be sent to at least one of the smart device 106, computer 118, or server 122. The at least one response may further include, at block 408, warming the wound 104 to the therapeutic temperature zone if the wound is hypothermic and the wound temperature is outside of the therapeutic temperature zone. Warming of the wound 104 is achieved when the system 100 instructs the heater 114 (or heater 214) to turn on and remain on until the wound 104 is warmed to a normal temperature within the therapeutic zone, as detected by the heat sensor 110 and/or one or more temperature probes 116. The ability to warm the wound 104 within the therapeutic temperature zone using heater 114 or 214 improves existing wound monitoring systems by providing a mechanism to shorten the optimal wound healing trajectory. In this regard, maintaining the wound 104 at an appropriate temperature zone is vital to continued healing of the wound 104. Since faster healing results in a shorter length of stay, there is less opportunity for complications to arise during the healing process.

Block 406 may further include triggering at least one response if the wound humidity is outside of a therapeutic humidity zone (e.g., the wound is too dry). In some embodiments, the at least one response includes sending an alert if the wound humidity is outside of the therapeutic humidity zone. The alert may be a text-based message indicating that the wound 104 requires moistening, and that water or a sterile/aseptic liquid should be applied to the wound. In embodiments in which the body-mounted heater 214 is used, the at least one response may further include activating the misting device of the heater 214 to supply moisture to the wound 104.

At block 410, the system 100 instructs the heater 114 (or heater 214) to turn off once the wound temperature reaches the therapeutic zone. After heater 114 (or heater 214) turns off, the heat sensor 110 and/or one or more temperature probes 116 continue to detect the wound temperature. The at least one response may also include, at block 412, automatically documenting the set of wound features and wound temperature/humidity in an electronic medical record (EMR). The EMR may be included as part of database 124 or may be a separate database configured to transmit and/or receive data over the network 112.

The smart device 106, computer 118, and/or server 122 may maintain a machine learning algorithm or model which has been trained using a library of wound statistics to determine an optimal wound healing trajectory, predict at least one future wound condition, and/or output a wound healing recommendation. After the machine learning algorithm is trained, in operation, the camera 108, heat sensor 110, and/or one or more temperature probes 116 capture data (e.g., images, temperatures, and/or humidity) of the subject's 102 wound 104. The collected data is input to the trained machine learning algorithm at block 414, which outputs a determined optimal wound healing trajectory, a predicted at least one future wound condition based on the optimal wound healing trajectory, and/or a wound healing recommendation at block 416. Additional details regarding the determined optimal wound healing trajectory, the predicted at least one future wound condition, and/or the wound healing recommendation are described in greater detail below.

During training of the machine learning algorithm, subject volunteers or providers/care teams may provide a variety of wound images and wound data (e.g., temperature and/or humidity). The wound images and wound data may be pre-existing or may be captured using smart device 106 (e.g., using camera 108 and heat sensor 110). The captured data may be labeled based on the particular stage of healing (e.g., wound is one week old, wound is one month old) that a wound is at when an image or other sensor data is captured to generate training data. The labeled captured data may include a library of wound statistics that includes the various wound images at various stages of healing and the associated set of wound features for each wound image at each stage of healing. Thus, the library of wound statistics may include but is not limited to at least one of similar wound features, similar wound temperatures/humidities, and stages of a wound healing. The library of wound statistics may further include but is not limited to at least one of subject demographics, subject comorbidities, or therapies and procedures that impact wound healing. The library of wound statistics may be stored in database 124. As such, a variety of training data may be captured to train the machine learning algorithm without overfitting the model to a narrow set of training data. The smart device 106, computer 118, and/or server 122 may then train the machine learning algorithm with the library of wound statistics to determine the optimal wound healing trajectory, predict the at least one future wound condition, and/or output the wound healing recommendation, as disclosed in further detail below. Use of the library of wound statistics to train the machine learning algorithm improves upon existing wound monitoring systems because the accuracy of the determined optimal wound healing trajectory and the predicted at least one future wound condition improves over time. That is, as additional wounds are tracked and assessed by the system 100, the data obtained from these additional wounds is added to the library of wound statistics, thereby increasing the amount of data from which the machine learning algorithm can make determinations and/or predictions.

As wound images and other sensor data is captured by the camera 108, heat sensor 110, and/or one or more temperature probes 116, the captured wound images and sensor data may be transmitted to the smart device 106, computer 118, and/or server 122. The smart device 106, computer 118, and/or server 122 may receive the images from the camera 108 and the data from the heat sensor 110 and/or one or more temperature probes 116 and may train and maintain a machine learning algorithm to determine the optimal wound healing trajectory, the at least one future wound condition, and/or the wound healing recommendation based on the received images and sensor data, as disclosed herein. During training, the smart device 106, computer 118, and/or server 122 may receive training data (e.g., the library of wound statistics) and may train the machine learning algorithm based on the received training data using supervised learning techniques, for example. After the machine learning algorithm is trained with the library of wound statistics, during real-time operation, the smart device 106, computer 118, and/or server 122 receive real-time data from the camera 108, heat sensor 110, and/or one or more temperature probes 116 (e.g., the set of wound features) and input the received data (e.g., the set of wound features) into the trained algorithm for processing using the trained algorithm. The smart device 106, computer 118, and/or server 122 may determine the optimal wound healing trajectory, predict the at least one future wound condition, and/or output the wound healing recommendation based on the received data.

As used herein, “machine learning algorithm” refers to a set of mathematical and statistical techniques and procedures that enable computers, such as smart device 106, computer 118, and/or server 122, to automatically learn and improve their performance on a specific task or problem without being explicitly programmed. The term “machine learning algorithm”, as used herein, may refer to various types including but not limited to supervised learning, unsupervised learning, and/or reinforcement learning. Specific examples of machine learning algorithms used herein may include but are not limited to linear regression, decision trees, support vector machines, k-means clustering, and/or neural networks.

As used herein, “real-time operation” refers to operation of the system 100 to determine the optimal wound healing trajectory, predict the at least one future wound condition, and/or output the wound healing recommendation after the machine learning model has been trained. As used herein, “real-time data” refers to data received from the camera 108, heat sensor 110, and/or one or more temperature probes 116 during real-time operation.

The machine learning algorithm described above may be included in the memory of the smart device 106, computer 118, and/or server 122 as a program module. The program module may be in the form of operating systems, application program modules, and other program modules stored in the memory. Such a program may include, but is not limited to, routines, subroutines, programs, objects, components, data structures and the like for performing specific tasks or executing specific data types. For example, the program module may include programming for causing the processor of the smart device 106 and/or computer 118 to receive sensor data from the camera 108, the heat sensor 110, and/or the one or more temperature probes 116. In addition, the program module may include programming for causing the processor of smart device 106 and/or computer 118 to pre-process the sensor data from the camera 108, the heat sensor 110, and/or the one or more temperature probes 116 before the data is input into the machine learning algorithm for processing using machine learning. Pre-processing of the sensor data may include combining the different types of data into a single training example to be input into the machine learning algorithm. Pre-processing of the sensor data may also include formatting data, filtering data, cropping images, and the like. Furthermore, the program module may include programming for causing the processor of the smart device 106 and/or computer 118 to train the machine learning algorithm using supervised learning techniques, for example. In addition, the program module may include programming for causing the processor of the smart device 106 and/or computer 118 to determine the optimal wound healing trajectory, predict the at least one future wound condition, and/or output the wound healing recommendation.

The set of wound features and wound temperature data is input into the machine learning algorithm for processing using machine learning, and the machine learning algorithm is configured to determine an optimal wound healing trajectory at block 414. In other words, the machine learning algorithm is configured to output a variety of information associated with the optimal wound healing trajectory, including but not limited to, the healing stage of the wound 104, the time remaining for the wound 104 to heal, and the therapeutic temperature zone for optimal healing of the wound 104. The optimal wound healing trajectory is further transmitted to the smart device 106 or computer 118 such that the care team of subject 102 can view the optimal wound healing trajectory on the display 107 of the smart device 106 and/or the display 120 of the computer 118. In this regard, the machine learning algorithm may include as output a representation of the optimal wound healing trajectory in the form of a text-based message.

Based on the optimal wound healing trajectory, the machine learning algorithm further predicts at least one future wound condition. The at least one future wound condition may also be described as a future set of wound features. As such, the at least one future wound condition includes but is not limited to a future temperature, future humidity, future size, future shape, future color, and/or future depth of the wound 104. The at least one future wound condition is further transmitted to the smart device 106 or computer 118 such that the care team of subject 102 can view the at least one future wound condition on the display 107 of the smart device 106 and/or the display 120 of the computer 118. In this regard, the machine learning algorithm may include as output a representation of the at least one future wound condition in the form of a 2-D or 3-D computer generated image.

At block 416, the machine learning algorithm is also configured to output a wound healing recommendation as a result of an analysis of the set of wound features and wound temperature data. The wound healing recommendation includes but is not limited to best practices for care of the wound or risks associated with healing of the wound. The wound healing recommendation is further transmitted to the smart device 106 or computer 118 such that the care team of subject 102 can view the wound healing recommendation on the display 107 of the smart device 106 and/or the display 120 of the computer 118. In this regard, the machine learning algorithm may include as output a representation of the wound healing recommendation in the form of a text-based message.

As discussed above, best practices for wound care and risks for wound healing are suggested at block 416, and the provider/care team may implement these suggestions to assist compliance with the optimal would healing trajectory determined at block 414. In this regard, the set of wound features and wound temperature change over time, and the system 100 continues to track and assess the wound 104 as these changes occur. That is, the system 100 is configured to adjust to the current state of the wound. For example, the wound 104 may eventually enter a healing zone which corresponds to a different therapeutic temperature zone compared to the therapeutic temperature zone when initial tracking and assessing of the wound 104 began. The healing zone may be determined based on the temperatures detected by the heat sensor 110 and one or more temperature probes 116 reaching a temperature change threshold, which may be different for different types of wounds.

Thus, at block 418, the system 100 continues to trigger at least one response if the wound temperature is outside of a new or healing therapeutic temperature zone. In some embodiments, the at least one response includes sending an alert if the wound temperature is outside of the healing therapeutic temperature zone. The alert may be sent to at least one of the smart device 106, computer 118, or server 122. The at least one response may further include warming the wound to the healing therapeutic temperature zone using the heater 114 (or heater 214) as described above with respect to block 406.

At block 420, the at least one response may additionally include automatically updating the set of wound features and wound temperature in the EMR of the subject 102. In other words, the analytics for the wound 104 are updated in near time to assist compliance with the optimal healing time. As used herein, “near time” means as close to real time as possible. In the context of updating the EMR, near time is as fast as the EMR can incorporate the updated analytics.

Over time, the system 100 may determine that the wound 104 has progressed through the end of the optimal healing trajectory based on the set of wound features and wound temperature. That is, the system 100 may determine that the set of wound features and wound temperature have reached a threshold indicating the optimal healing trajectory has been met. Thus, at block 422, the system 100 collects and reports the clinical course and outcome of the wound 104 to national, local, and institutional wound registries.

A method for tracking and assessing a burn wound using system 100 discussed above will now be described with reference to FIG. 5. The method illustrated in FIG. 5 is similar to the method illustrated in FIG. 4 except that the method illustrated in FIG. 5 is specifically directed to burn wounds. In this regard, some steps of the method of FIG. 5 will be described in the following with reference to the method of FIG. 4 in the interest of brevity. In some embodiments, various steps of the method may be performed by smart device 106, computer 118, and/or remote server 122. The various steps may be performed on separate computing devices and control systems or the steps may all be performed by a single processor.

At block 500, a subject (e.g., subject 102) experiences a burn wound in the field. The burn wound may be a thermal, chemical, or electrical burn, for example.

At block 502, a set of burn wound features is captured using smart device 106. To obtain the set of burn wound features, the system 100 identifies the burn wound (e.g., wound 104) from an image output by the camera 108. Identification of the burn wound is performed in a substantially similar manner as the identification of the wound discussed above with respect to block 404 of the method illustrated in FIG. 4. Thus, the processor of smart device 106 or computer 118 may process the image to identify the burn wound boundaries based on pixel locations, color change thresholds, changes in height or depth, and/or any other suitable computer vision processing techniques. Once the burn wound is identified by the image output by camera 108, the set of burn wound features within the burn wound boundaries are obtained by the system 100 based on the image. The set of burn wound features is obtained in a substantially similar manner as the set of wound features described above with respect to block 404 of the method illustrated in FIG. 4. Thus, the set of burn wound features may include temperature, humidity, size, shape, color, and/or depth.

The set of burn wound features thus being obtained are provided to a service, such as, for example, a local emergency medical service (EMS), at block 504. For example, the set of burn wound features are transmitted to the smart device 106 or computer 118 such that the EMS can view the set of burn wound features on the display 107 of the smart device 106 or the display 120 of the computer 118. The set of burn wound features may also be stored in the memory of the smart device 106 and/or the memory of the computer 118 for subsequent retrieval, manipulation, and/or management.

At block 506, the local EMS triages the burn wound using the smart device 106. Triaging of the burn wound is based on sensor data from one or more sensors (e.g., camera 108, which may be a depth camera, or heat sensor 110, which may be a thermal camera). Triaging is performed so that the preliminary extent of the burn can be determined prior to admission at an emergency room (ER).

At block 508, the set of burn wound features and sensor data are input into a machine learning algorithm for processing using machine learning and an optimal burn wound healing trajectory is suggested via the machine learning algorithm. In other words, the set of burn wound features and sensor data are processed with the machine learning algorithm, as implemented by the smart device 106 and/or computer 118, to determine an optimal burn wound healing trajectory. The optimal burn wound healing trajectory is transmitted to the smart device 106 or computer 118 such that the care team of subject 102 can view the optimal burn wound healing trajectory on the display 107 of the smart device 106 and/or the display 120 of the computer 118. Similar to the method illustrated in FIG. 4, the set of burn wound features and sensor data is also processed with the machine learning algorithm to predict at least one future burn wound condition based on the optimal wound healing trajectory. The at least one future burn wound condition is also displayed on the display 107 of the smart device 106 and/or the display 120 of the computer 118. As discussed above with respect to the method illustrated in FIG. 4, the machine learning algorithm is trained with a library of burn wound statistics. Similar to the library of wound statistics discussed above, the library of burn wound statistics may include but is not limited to similar burn wound features, similar burn wound temperatures/humidities, and stages of a burn wound healing. The library of burn wound statistics may further include, but is not limited to subject demographics, subject comorbidities, or therapies and procedures that impact burn wound healing.

Additional processing of the set of burn wound features and sensor data occurs at block 510, where the set of burn wound features and sensor data are input into the machine learning algorithm for processing using machine learning and the machine learning algorithm outputs a burn wound healing recommendation. The burn wound healing recommendation may include but is not limited to at least one of best practices for care of the burn wound and/or risks associated with healing of the burn wound.

In some instances, the machine learning algorithm may output a burn wound healing recommendation that the best practice for care of the burn wound is to undergo a skin graft surgery. In the event that a skin graft is suggested, the burn wound is assessed post-operatively using the system 100 at block 512. That is, the skin graft wound is assessed using system 100. This post-operative assessment may include repeating block 502 to capture a set of skin graft wound features. This post-operative assessment also includes a temperature and/or humidity assessment via the heat sensor 110 (e.g., thermal camera) and/or the one or more temperature probes 116. As such, the heat sensor and/or one or more temperature probes 116 may be used as described in blocks 400 and 402 described above to detect, in real time, a temperature and/or humidity of the skin graft from the one or more sensors.

Once the one or more sensors (e.g., heat sensor 110 and/or one or more temperature probes 116) are detecting real time temperature, the system 100 may trigger at least one response if the skin graft temperature is outside of a therapeutic temperature zone at block 514. As discussed above with respect to the method of FIG. 4, triggering the at least one response may include sending an alert if the skin graft temperature is outside of the therapeutic temperature zone. Triggering the at least one response may also include warming the skin graft to the therapeutic temperature zone if the skin graft temperature is outside of the therapeutic temperature zone. Warming of the skin graft is performed by the heater 114 (or heater 214) as described above.

At block 516, triggering the at least one response may also include identifying potential early skin graft failure based on the temperature assessment and communicating the potential early skin graft failure to the provider/care team of the subject. That is, the machine learning algorithm may identify the potential early skin graft failure based on the temperature detected by the one or more sensors (e.g., heat sensor 110 and/or one or more temperature probes 116).

At block 518, triggering the at least one response may additionally include the machine learning algorithm suggesting interventions for responding to the potential early skin graft failure, such that the interventions can be captured by documentation of the provider/care team of the subject. For example, the interventions may be input and stored in the subject's EMR.

At block 520, triggering the at least one response may further include updating the analytics for the skin graft wound in near time to assist compliance with the optimal healing time.

Over time, the system 100 may determine that the skin graft has progressed through the end of the optimal healing trajectory. That is, the system 100 may determine that the set of skin graft wound features and temperature have reached a threshold indicating the optimal healing trajectory has been met. Thus, at block 522, the system 100 collects and reports the clinical course and outcome of the skin graft wound to national, local, and institution burn wound registries.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

It should now be understood that embodiments described herein are directed to methods to track and assess a wound. As discussed above, a wound is identified from an image and a set of wound features is identified based on the image. A temperature and/or humidity of the wound is detected in real time and a response is triggered if the wound temperature is outside of a therapeutic temperature zone and/or a therapeutic humidity zone. Real time temperature detection described herein improves existing wound monitoring systems because decisions affecting wound healing can be made more quickly. The response may include activating a heater to warm the wound. The ability to warm the wound using a heater improves existing wound monitoring systems by providing a mechanism to shorten an optimal wound healing trajectory. The set of wound features and wound temperature are input into a machine learning algorithm for processing using machine learning, and the optimal wound healing trajectory is determined. The machine learning algorithm is trained on a library of wound statistics. Use of the library of wound statistics improves upon existing wound monitoring systems because the accuracy of determining the optimal wound healing trajectory improves over time. Moreover, the embodiments disclosed herein track and assess a wound objectively because the tracking and assessing is based on objective factors. For example, the tracking and assessing described herein may be based on wound location, size, shape, color, depth, temperature, humidity, etc.

Further aspects of the invention are provided by the subject matter of the following clauses.

A method for tracking and assessing a wound, the method including identifying a wound from an image output by a camera; identifying a set of wound features based on the image; detecting a temperature of the wound from at least one heat sensor; triggering at least one response if the wound temperature is outside of a therapeutic temperature zone; processing the set of wound features and wound temperature using machine learning; determining an optimal wound healing trajectory based on the set of wound features and the wound temperature using machine learning; predicting at least one future wound condition based on the optimal wound healing trajectory using machine learning; and displaying the optimal wound healing trajectory and the at least one future wound condition on a display.

The method of any previous clause, wherein at least one of the at least one heat sensor is a thermal camera.

The method of any previous clause, wherein at least one of the at least one heat sensor is a temperature probe placed around a perimeter of the wound.

The method of any previous clause, wherein triggering the at least one response comprises sending an alert if the wound temperature is outside of the therapeutic temperature zone.

The method of any previous clause, wherein triggering the at least one response comprises warming the wound to the therapeutic temperature zone if the wound temperature is outside of the therapeutic temperature zone.

The method of any previous clause, wherein triggering the at least one response comprises documenting the set of wound features and the wound temperature in an electronic medical record.

The method of any previous clause, further including training the machine-learning algorithm with a library of wound statistics.

The method of any previous clause, wherein the library of wound statistics includes at least one of: similar wound features, similar wound temperatures, stages of wound healing, subject demographics, subject comorbidities, or therapies and procedures that impact wound healing.

The method of any previous clause, further including outputting a wound healing recommendation as a result of an analysis of the set of wound features and wound temperature using machine learning.

The method of any previous clause, wherein the wound healing recommendation includes at least one of: best practices for care of the wound or risks associated with healing of the wound.

The method of any previous clause, wherein detecting the temperature of the wound from at least one heat sensor is performed in real time.

The method of any previous clause, further including detecting a humidity of the wound from the at least one heat sensor.

A system for tracking and assessing a wound, the system including a display; a camera configured to output image data; at least one heat sensor configured to output temperature data; a memory containing a machine readable medium including machine executable code having instructions stored thereon; a controller communicatively coupled to the display, camera, at least one heat sensor, and memory, the controller including one or more processors and configured to execute the machine executable code to cause the controller to: identify a wound and a set of wound features from the image data; detect a temperature of the wound from the at least one heat sensor; trigger at least one response if the wound temperature is outside of a therapeutic temperature zone; process the set of wound features and wound temperature using machine learning; determine an optimal wound healing trajectory based on the set of wound features and the wound temperature using machine learning; predict at least one future wound condition based on the optimal wound healing trajectory using machine learning; and display the optimal wound healing trajectory and the at least one future wound condition on the display.

The system of any previous clause, wherein the at least one heat sensor is a thermal camera.

The system of any previous clause, wherein the at least one heat sensor is one or more temperature probes placed around a perimeter of the wound.

The system of any previous clause, further including a heater configured to supply at least one type of heat to the wound, wherein the controller is communicatively coupled to the heater.

The system of any previous clause, wherein the at least one type of heat is radiant heat or convection heat.

The system of any previous clause, wherein the heater is an electrically powered heating sleeve placed over the wound.

The system of any previous clause, wherein the controller is further configured to activate the heater until the wound temperature reaches the therapeutic temperature zone.

The system of any previous clause, wherein detecting the temperature of the wound from the at least one heat sensor is performed in real time.

A method for tracking and assessing a burn wound, the method including identifying a burn wound from an image output by a camera; capturing a set of burn wound features based on the image; triaging the burn wound based on sensor data from one or more sensors; processing the burn wound features and sensor data with a machine learning algorithm to determine an optimal burn wound healing trajectory, wherein the machine learning algorithm is trained with a library of burn wound statistics; predicting at least one future burn wound condition based on the optimal wound healing trajectory with the machine learning algorithm; and displaying the optimal burn wound healing trajectory and the at least one future burn wound condition on a display.

The method of any previous clause, wherein the one or more sensors includes at least one of a depth camera or a thermal camera.

The method of any previous clause, wherein the library of burn wound statistics includes at least one of: similar burn wound features, stages of burn wound healing, subject demographics, subject comorbidities, or therapies and procedures that impact burn wound healing.

The method of any previous clause, wherein processing the burn wound features and sensor data includes inputting the burn wound features and sensor data into the machine learning algorithm and outputting a burn wound healing recommendation.

The method of any previous clause, wherein the burn wound healing recommendation includes at least one of: best practices for care of the burn wound care or risks associated with healing of the burn wound.

The method of any previous clause, wherein the burn wound is a skin graft and the method further comprises detecting, in real time, a temperature of the skin graft from the one or more sensors and triggering at least one response if the skin graft temperature is outside of a therapeutic temperature zone.

The method of any previous clause, wherein triggering the at least one response comprises: sending an alert if the skin graft temperature is outside of the therapeutic temperature zone or warming the skin graft to the therapeutic temperature zone if the skin graft temperature is outside of the therapeutic temperature zone.

The method of any previous clause, further including reporting at least one of the burn wound features, sensor data, or optimal burn wound healing trajectory to one or more burn wound registries.

A non-transitory, processor-readable storage medium, including: one or more programming instructions stored thereon, the one or more programming instructions, when executed, causing a processing device to carry out the method according to any one of the previous clauses.

What is claimed is:

1. A method for tracking and assessing a wound, the method comprising:
identifying a wound from an image output by a camera;
identifying a set of wound features based on the image;
detecting a temperature of the wound from at least one heat sensor;
triggering at least one response if the wound temperature is outside of a therapeutic temperature zone;
processing the set of wound features and wound temperature using machine learning;
determining an optimal wound healing trajectory based on the set of wound features and the wound temperature using machine learning;
predicting at least one future wound condition based on the optimal wound healing trajectory using machine learning; and
displaying the optimal wound healing trajectory and the at least one future wound condition on a display.

2. The method according to claim 1, wherein at least one of the at least one heat sensor is a thermal camera.

3. The method according to claim 1, wherein at least one of the at least one heat sensor is a temperature probe placed around a perimeter of the wound.

4. The method according to claim 1, wherein triggering the at least one response comprises sending an alert if the wound temperature is outside of the therapeutic temperature zone.

5. The method according to claim 1, wherein triggering the at least one response comprises warming the wound to the therapeutic temperature zone if the wound temperature is outside of the therapeutic temperature zone.

6. The method according to claim 1, wherein triggering the at least one response comprises documenting the set of wound features and the wound temperature in an electronic medical record.

7. The method according to claim 1, further comprising training a machine learning algorithm with a library of wound statistics.

8. The method according to claim 7, wherein the library of wound statistics includes at least one of: similar wound features, similar wound temperatures, stages of wound healing, subject demographics, subject comorbidities, or therapies and procedures that impact wound healing.

9. The method according to claim 1, further comprising outputting a wound healing recommendation as a result of an analysis of the set of wound features and wound temperature with the machine learning.

10. The method according to claim 9, wherein the wound healing recommendation includes at least one of: best practices for care of the wound or risks associated with healing of the wound.

11. The method according to claim 1, wherein detecting the temperature of the wound from at least one heat sensor is performed in real time.

12. The method according to claim 1, further comprising detecting a humidity of the wound based on one or more signals obtained from the at least one heat sensor.

13. A system for tracking and assessing a wound, the system comprising:
a display;
a camera configured to output image data;
at least one heat sensor configured to output temperature data;
a memory containing a machine readable medium including machine executable code having instructions stored thereon;
a controller communicatively coupled to the display, camera, at least one heat sensor, and memory, the controller including one or more processors and configured to execute the machine executable code to cause the controller to:
identify a wound and a set of wound features from the image data;
detect a temperature of the wound from the at least one heat sensor;
trigger at least one response if the wound temperature is outside of a therapeutic temperature zone;
process the set of wound features and wound temperature using machine learning;
determine an optimal wound healing trajectory based on the set of wound features and the wound temperature using machine learning;
predict at least one future wound condition based on the optimal wound healing trajectory using machine learning; and
display the optimal wound healing trajectory and the at least one future wound condition on the display.

14. The system according to claim 13, wherein the at least one heat sensor is a thermal camera.

15. The system according to claim 13, wherein the at least one heat sensor is one or more temperature probes placed around a perimeter of the wound.

16. The system according to claim 13, further comprising a heater configured to supply at least one type of heat to the wound, wherein the controller is communicatively coupled to the heater.

17. The system according to claim 16, wherein the at least one type of heat is radiant heat or convection heat.

18. The system according to claim 16, wherein the heater is an electrically powered heating sleeve placed over the wound.

19. The system according to claim 16, wherein the controller is further configured to activate the heater until the wound temperature reaches the therapeutic temperature zone.

20. The system according to claim 16, wherein detecting the temperature of the wound from the at least one heat sensor is performed in real time.
